(19) United States
(12) Patent Application Publication
(10) Pub. No.: US 2017/0114128 A1
(43) Pub. Date: Apr. 27, 2017

(54) USE OF TGF-BETA ANTAGONISTS OF TREAT TYPE-2 DIABETES

(71) Applicant: ALBERT EINSTEIN COLLEGE OF MEDICINE, INC., Bronx, NY (US)

(72) Inventor: Dongsheng Cai, Larchmont, NY (US)

(73) Assignee: Albert Einstein College of Medicine, Inc., Bronx, NY (US)

(21) Appl. No.: 15/318,022

(22) PCT Filed: Jul. 17, 2015
(86) PCT No.: PCT/US2015/040833

§ 371 Date: Dec. 12, 2016

Related U.S. Application Data
(60) Provisional application No. 62/026,126, filed on Jul. 18, 2014.

(51) Int. Cl. C07K 16/22; C07K 14/71; C07K 16/28

(52) U.S. Cl. CPC C07K 16/22; C07K 16/2881; C07K 16/2863; C07K 14/71; C07K 2317/31; C07K 2317/76; C07K 2319/00; A61K 38/00

(57) ABSTRACT

Method and compositions for treating type-2 diabetes in a subject are provided comprising administering to the subject an amount of an inhibitor of a TGF-beta.

### CROSS-REFERENCE TO RELATED APPLICATIONS

This application claims benefit of U.S. Provisional Application No. 62/026,126, filed Jul. 18, 2014, the contents of which are hereby incorporated by reference.

### STATEMENT OF GOVERNMENT SUPPORT

This invention was made with government support under grant numbers R01 DK078750, R01 AG031774, R01 HL113180, awarded by the National Institutes of Health. The government has certain rights in the invention.

### BACKGROUND OF THE INVENTION

Throughout this application various publications are referred to in square brackets. Full citations for these references may be found at the end of the specification. The disclosures of these publications, and all patents, patent application publications and books referred to herein, are hereby incorporated by reference in their entirety into the subject application to more fully describe the art to which the subject invention pertains.

Type-2 diabetes (T2D), one of most prevalent chronic diseases in developed societies, is initiated by the induction of glucose intolerance, a pre-diabetic state of hyperglycemia that is frequently caused by insulin resistance in peripheral tissues such as liver and muscles. Over decades, many research activities have been focusing on peripheral tissues in order to depict the mechanisms of glucose intolerance and insulin resistance, and indeed, multiple models of molecular mechanisms were elucidated (1-4). Despite these important progresses, there is still a critical lack of successful solutions for stopping T2D epidemic, perhaps implicating that additional mechanisms remain to be unveiled. Interestingly, recent research advance in neuroendocrinology has increasingly suggested that the central nervous system (CNS), in particular the comprised hypothalamus, has explicit impacts on glucose homeostasis (5-9), and these effects can be dissociable from the role of the hypothalamus in regulating body weight which has been extensively studied over the past decades (10,11). However, it remains unexplored if the brain could casually translate certain pro-diabetic etiology, such as obesity and aging, into the development of T2D. Of note, hypothalamic inflammation was recently demonstrated to occur in not only obesity (12-22) but also aging (23-25). In general, hypothalamic inflammation in obesity or aging is attributed to an atypical format of pro-inflammatory NF-κB activation (12-14,18-20,23-27); yet, the causes and characteristics of this atypical inflammation are not known or understood.

The present invention addresses the need for more precise therapies for controlling T2D and reducing the development of T2D by targeting TGF-beta in the central nervous system.

### SUMMARY OF THE INVENTION

A method of treating type-2 diabetes in a subject comprising administering to the subject an amount of an inhibitor of TGF-beta activity, in a manner effective to enter the central nervous system (CNS) of a subject, effective to treat type-2 diabetes in a subject.

A method of reducing development of type-2 diabetes in a subject comprising administering to the subject an amount of an inhibitor of TGF-beta activity, in a manner effective to enter the central nervous system (CNS) of a subject, effective to reduce development of type-2 diabetes in a subject.

An assay for identifying a treatment for type-2 diabetes comprising contacting a TGF-beta with a small organic molecule and determining if the small organic molecule inhibits activity of the TGF-beta as compared to a non-binding placebo, and positively identifying a small organic molecule which does inhibit activity of the TGF-beta as compared to a non-binding placebo as a treatment for type-2 diabetes.

An assay for identifying a treatment for type-2 diabetes comprising contacting a TGF-beta receptor with a small organic molecule and determining if the small organic molecule inhibits activity of the TGF-beta receptor as compared to a non-binding placebo, and positively identifying a small organic molecule which does inhibit activity of the TGF-beta receptor as compared to a non-binding placebo as a treatment for type-2 diabetes.

A method of reducing glucose intolerance in a subject comprising administering to the subject an amount of an inhibitor of TGF-beta activity, in a manner effective to enter the central nervous system (CNS) of a subject, effective to reduce glucose intolerance in a subject.

A method of reducing insulin intolerance in a subject comprising administering to the subject an amount of an inhibitor of TGF-beta activity, in a manner effective to enter the central nervous system (CNS) of a subject, effective to reduce insulin intolerance in a subject.

### BRIEF DESCRIPTION OF THE DRAWINGS

FIG. 1A-1K. Brain TGF-β1 excess induces systemic glucose disorder. Male C57BL/6 mice fed on a HFD vs. chow for indicated weeks (W) (a, c), and chow-fed C57BL/6 mice at the ages of indicated months (M) (b, d) were analyzed for Tgfb1 mRNA in the hypothalamus (a, b) or TGF-β1 concentrations in the CSF (c, d). C57BL/6 mice were injected with vehicle (Veh) vs. TGF-β1 at the indicated doses (e) or 4 ng (f-j) and examined with GTT (e), ITT (f) or insulin clamp (g-j). Inserted bars (e, f) show the area under curve (AUC) of GTT (unit: mg dl-1×120 min, ×103) and ITT (% of control). Glucose infusion rate (GIR) (g), rate of glucose disposal (Rd) (h), and hepatic glucose production (GP) (i-j) in the clamp experiment were determined *P<0.05, **P<0.01, ***P<0.001; n=4 (a-d), 7-9 (e, f), and 5 (g-j) mice per group. Error bars reflect mean ±SEM.

FIG. 2A-2D. Astrocyte-specific TGF-β1 transgenic expression leads to glucose disorder. Co-immunostaining of TGF-β1 with GFAP (a) or NeuN (b) of hypothalamic sections generated from male GFAP-Tgfb1tg/− mice (G-Tgfb1tg/−) and littermate controls (Con). Images show a sub-area in the MBH, and nuclear staining by DAPI revealed cells in sections. Scale bar=50 μm. Food intake (c), body weight (d), GTT (e) and ITT (f) were determined in chow-fed G-Tgfb1tg/− and littermate Con. Inserted bar graphs show the area under curve (AUC) values of GTT (unit: mg dl-1×120 min, ×103) and ITT (% of Con). *P<0.05, **P<0.01; n=7 8 mice per group. Error bars reflect mean ±SEM.

FIG. 3A-3D. Cell-specific TGF-β1 inhibition reduces diet-induced glucose disorder.

Adult male GFAP-Tgfb1lox/lox mice (G-Tgfb11/1; a, b), POMC-Tgfbr2lox/lox mice (P-Tgfbr21/1; c, d) and matched littermate controls (Con) were fed on a HFD for 3 weeks and examined for GTT (a, c), ITT (b, d). *P<0.05, n=6-8 mice per group. Error bars reflect mean ±SEM.

FIG. 4A-4D. Effect of TGF-β1 excess on hypothalamic inflammation. (a) Male C57BL/6 mice were injected with TGF-β1 vs. vehicle (Veh), and hypothalami were collected for Western blots. Western blot data represent 4 mice per group. (b) Hypothalami of male Tgfb1 +/− and littermate WT mice were collected and analyzed for mRNA levels of indicated genes. (c, d) Male T1r4−/− mice and littermate WT were injected with TGF-β1 vs. vehicle, and subjected to GTT (e) or ITT (f). *P<0.05, **P<0.01, ns, non-significant; n=4 (b) and 8-10 (c-f) mice per group. Error bars reflect mean ±SEM.

FIG. 5A-5G. Effects of TGF-β1 on hypothalamic RNA SGs and IκBα mRNA decay. (a, b) Male C57BL/6 mice were injected with TGF-β1 (4 ng) vs. vehicle (Veh), and hypothalami were harvested for measuring mRNA levels of SGs/PBs components (a) or HuR immunostaining (b). Nuclear staining by DAPI revealed cells in sections. Images show a representative sub-area of the MBH. Scale bar=10 nm. (c, d) GT1-7 cells were treated with TGF-β1 (10 ng/ml) for the indicated durations and were harvested for measuring IκBα mRNA levels. (e) Male C57BL/6 mice were injected with TGF-β1 (4 ng) vs. vehicle (Veh), and hypothalami were harvested for measuring mRNA levels of IκBα. (f-g) Male C57BL/6 mice received MBH injection of lentiviral dominant-negative IκBα vs. control (Con), and were injected with TGF-β1 vs. vehicle. Mice were killed for Western blots (f), or examined with ITT (g). Bar graph shows the area under curve (AUC) values of ITT. *P<0.05, **P<0.01, ***P<0.001, n=4 mice per group (a, e), and n=4 samples per group (c, d), and n=5-8 mice per group (g). Error bars reflect mean ±SEM. AU: arbitrary unit.

FIG. 6A-6E. Hypothalamic TGF-β and RNA SGs/PBs link aging to glucose disorders. Male C57BL/6 mice (a, b) and Tgfb1+/vs. WT mice (Con) (c-f) were analyzed at young vs. middle-aged age (2 vs. 15 months old). Hypothalamic mRNA levels of SGs/PBs components (a) and HuR immunostaining (b), food intake (c), body weight (d), and blood glucose in GTT (e) and ITT (f) were analyzed. Scale bar=10 μm (b). *P<0.05, **P<0.01, ***P<0.001; n=4 mice per group (a, b), and n=5-8 mice per group (c-f). Error bars reflect mean ±SEM.

FIG. 7A-7H: A single injection of SB431542 in the third ventricle significantly reduced glucose intolerance (FIG. 7a&b) and insulin intolerance (FIG. 7c&d). FIG. 7e-h, show aging was associated with impairment of glucose tolerance and insulin tolerance in control group. However, hypothalamic third-ventricle injection of SB431542 led to significant reductions in glucose and insulin intolerance (FIG. 7e-h).

FIG. 8: Possible pathways explaining action of TGF-beta.

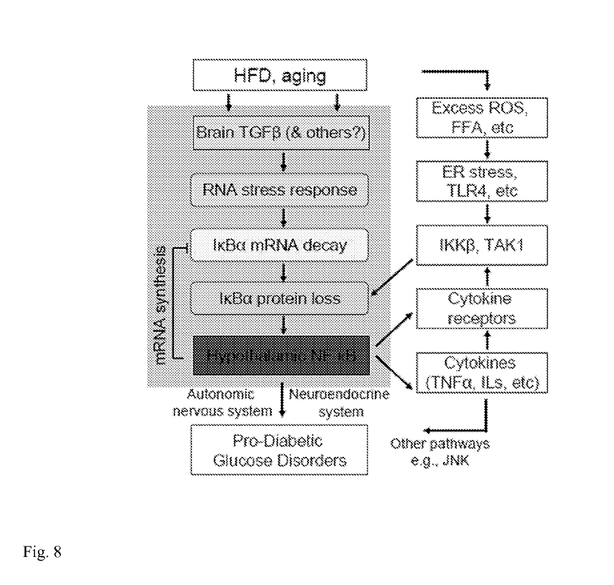

### DETAILED DESCRIPTION OF THE INVENTION

Herein it is described that the brain can directly induce pre-diabetic glucose disorder through the local, excessive effect of transforming growth factor-β (TGF-β), a cytokine which is often overproduced during inflammation and has mixed biological functions (28). Mechanistically, brain TGF-β excess induces hypothalamic RNA stress granules to enhance IκBα mRNA decay which activates hypothalamic NF-κB atypically, and thus mediates a hypothalamic inflammatory basis in co-linking obesity and aging to T2D development.

As used herein, to treat type 2 diabetes in a subject who has type 2 diabetes means to stabilize, reduce, ameliorate or eliminate a sign or symptom of type 2 diabetes in the subject.

A method of treating type-2 diabetes in a subject is provided comprising administering to the subject an amount of an inhibitor of TGF-beta activity, in a manner effective to enter the central nervous system (CNS) of a subject, effective to treat type-2 diabetes in a subject.

Also provided is a method of reducing development of type-2 diabetes in a subject comprising administering to the subject an amount of an inhibitor of TGF-beta activity, in a manner effective to enter the central nervous system (CNS) of a subject, effective to reduce development of type-2 diabetes in a subject.

Also provided is a method of reducing glucose intolerance in a subject comprising administering to the subject an amount of an inhibitor of TGF-beta activity, in a manner effective to enter the central nervous system (CNS) of a subject, effective to reduce glucose intolerance in a subject.

Also provided is a method of reducing insulin intolerance in a subject comprising administering to the subject an amount of an inhibitor of TGF-beta activity, in a manner effective to enter the central nervous system (CNS) of a subject, effective to reduce insulin intolerance in a subject.

In an embodiment of the methods, the inhibitor of TGF-beta activity binds to a TGF-beta molecule and inhibits activity thereof. In an embodiment, the inhibitor of TGF-beta activity binds to a TGF-beta receptor and inhibits activity thereof.

In an embodiment of the methods, the inhibitor of TGF-beta activity is administered directly to the CNS of the subject. Direct administration can be effected by any means known in the art, e.g. by injection, by cannula, via a drug-eluting CNS implant (the drug being the inhibitor of TGF-beta activity). In an embodiment of the methods, the inhibitor of TGF-beta is administered via nasal epithelia of the subject. In an embodiment of the methods, the inhibitor of TGF-beta is administered via an upper portion of the nasal epithelia of the subject.

In an embodiment of the methods, the inhibitor of TGF-beta is administered systemically but is able to cross the blood-brain barrier into the CNS of the subject. In an embodiment of the methods, the inhibitor of TGF-beta is administered encapsulated in a liposome. Preferably, such liposome can cross the blood-brain barrier into the CNS. In an embodiment of the methods, the liposome is glutathione-coated. In an embodiment of the methods, the inhibitor of TGF-beta is a bi-specific antibody that (i) (a) binds TGF-beta or (b) binds a TGF-beta receptor, and (ii) also binds a human transferrin receptor. In an embodiment of the methods, the inhibitor of TGF-beta is a bi-specific antibody that binds a TGF-beta molecule and also binds a human transferrin receptor. In an embodiment of the methods, the inhibitor of TGF-beta is a bi-specific antibody that binds a TGF-beta receptor, and also binds a human transferrin receptor. These permits transfer of the bi-specific antibody across the blood-brain barrier into the CNS where it can bind and inhibit TGF-beta or the TGF-beta receptor as appropriate. Preferably, the affinity of the bispecific antibody for the human transferrin receptor is a medium to low affinity (e.g. see Yu, Y. J. et al. Sci. Trans. Med. 3, 84ra44 (2011), hereby incorporated by reference), while the affinity for TGF-beta or the TGF-beta receptor is medium to high. Low affinity for a human transferrin receptor, as used herein, encompasses an IC₅₀ range of 10 nM to 1000 nM. In an embodiment, the low affinity is IC₅₀ range of 100 nM to 1000 nM. Also see US Patent Application No. 2012/0171120 (hereby incorporated by reference) for ranges of affinity for a human transferrin receptor encompassed by the present invention. In an embodiment of the methods, the inhibitor of TGF-beta is (i) a monoclonal anti-TGF-beta antibody conjugated to a lipoprotein receptor related protein receptor (LRP-1) binding-peptide of 8-40 amino acids, or (ii) a monoclonal anti-TGF-beta receptor antibody conjugated to a lipoprotein receptor related protein receptor (LRP-1) binding-peptide of 8-40 amino acids. Various LRP-1 binding peptides have been reported that can effect transfer of bound cargoes, such as an antibody, across the blood-brain barrier into the CNS.

In an embodiment of the methods, the inhibitor of TGF-beta activity is an isolated antibody, or an antigen-binding fragment of such an antibody. In an embodiment, the antibody is produced via the hand of man. In an embodiment, the administered antibody is a monoclonal antibody. In an embodiment, the antibody is a chimeric or humanized antibody. In an embodiment, the antibody is a human antibody that has been recombinantly produced outside of a human. As used herein, the term “antibody” refers to an intact antibody, i.e. with complete Fc and Fv regions. “Fragment” refers to any portion of an antibody, or portions of an antibody linked together, such as, in non-limiting examples, a Fab, F(ab)₂, a single-chain Fv (scFv), which is less than the whole antibody but which is an antigen-binding portion and which competes with the intact antibody of which it is a fragment for specific binding. As such a fragment can be prepared, for example, by cleaving an intact antibody or by recombinant means (e.g. scFv). See generally, Fundamental Immunology, Ch. 7 (Paul, W., ed., 2nd ed. Raven Press, N.Y. (1989), hereby incorporated by reference in its entirety). Antigen-binding fragments may be produced by recombinant DNA techniques or by enzymatic or chemical cleavage of intact antibodies or by molecular biology techniques. In some embodiments, a fragment is an Fab, Fab′, F(ab′)₂, F_{d}, Fᵥ, complementarity determining region (CDR) fragment, single-chain antibody (scFv), (a variable domain light chain (V_{L}) and a variable domain heavy chain (V_{H}) linked via a peptide linker. In an embodiment the linker of the scFv is 10-25 amino acids in length. In an embodiment the peptide linker comprises glycine, serine and/or threonine residues. For example, see Bird et al., Science, 242: 423-426 (1988) and Huston et al., Proc. Natl. Acad. Sci. USA, 85:5879-5883 (1988) each of which are hereby incorporated by reference in their entirety), or a polypeptide that contains at least a portion of an antibody that is sufficient to confer TGF-beta-specific or TGF-beta-receptor specific antigen binding on the polypeptide, including a diabody. From N-terminus to C-terminus, both the mature light and heavy chain variable domains comprise the regions FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md. (1987 and 1991)), Chothia & Lesk, J. Mol. Biol. 196:901-917 (1987), or Chothia et al., Nature 342:878-883 (1989), each of which are hereby incorporated by reference in their entirety). As used herein, the term “polypeptide” encompasses native or artificial proteins, protein fragments and polypeptide analogs of a protein sequence. A polypeptide may be monomeric or polymeric. As used herein, an F_{d} fragment means an antibody fragment that consists of the V_{H} and CH1 domains; an Fᵥ fragment consists of the V₁ and V_{H} domains of a single arm of an antibody; and a dAb fragment (Ward et al., Nature 341:544-546 (1989) hereby incorporated by reference in its entirety) consists of a V_{H} domain. In some embodiments, fragments are at least 5, 6, 8 or 10 amino acids long. In other embodiments, the fragments are at least 14, at least 20, at least 50, or at least 70, 80, 90, 100, 150 or 200 amino acids long. Since it is estimated that less than 1 in 1000 antibodies in the systemic circulation can cross the blood-brain barrier (BBB) (e.g. see world wide web.the-scientist.com/?articles.view/articleNo/37957/title/Pentetrating-the-brain/ by M. Scudellari, Nov. 1, 2013), modified antibodies are likely required which have higher rates of entry into the CNS across the BBB to achieve therapeutic levels if the antibodies are being administered systemically.

In an embodiment of the methods, the inhibitor of TGF-beta activity a synthetic fusion protein comprising a soluble TGF-beta receptor. In a further embodiment of the methods, the synthetic fusion protein comprises a portion having the sequence of a human immunoglobulin Fc. In an embodiment, the Fc portion has a sequence the same as the Fc portion of a human IgG. In an embodiment, the synthetic fusion protein is a TβRII-Fc. In an embodiment, the TβRII portion has a sequence the same as a human TβRII.

In an embodiment of the methods, the inhibitor of TGF-beta activity is a synthetic small organic compound. In an embodiment of the methods, the inhibitor of TGF-beta activity is a synthetic small organic compound of less than 1,500 Da. In an embodiment of the methods, the inhibitor of TGF-beta activity is an inhibitor of a TGF-beta receptor. In an embodiment of the methods, the inhibitor of TGF-beta activity is SB-431542 (4-[4-(1,3-benzodioxol-5-yl)-5-(2-pyridinyl)-1H-imidazol-2-yl]benzamide), A 83-01 (3-(6-Methyl-2-pyridinyl)-N-phenyl-4-(4-quinolinyl)-1H-pyrazole-1-carbothioamide), D 4476 (4-[4-(2,3-Dihydro-1,4-benzodioxin-6-yl)-5-(2-pyridinyl)-1H-imidazol-2-yl]benzamide), GW 788388 (4-[4-[3-(2-Pyridinyl)-1H-pyrazol-4-yl]-2-pyridinyl]-N-(tetrahydro-2H-pyran-4-yl)-benzamide), LY 364947 (4-[3-(2-Pyridinyl)-1H-pyrazol-4-yl]-quinoline), RepSox (2-(3-(6-Methylpyridine-2-yl)-1H-pyrazol-4-yl)-1,5-naphthyridine), SB 505124 (2-[4-(1,3-Benzodioxol-5-yl)-2-(1,1-dimethylethyl)-1H-imidazol-5-yl]-6-methyl-pyridine), SB 525334 (6-[2-(1,1-Dimethylethyl)-5-(6-methyl-2-pyridinyl)-1H-imidazol-4-yl]quinoxaline), or SD 208 (2-(5-Chloro-2-fluorophenyl)-4-[(4-pyridyl)amino]pteridine). In an embodiment of the methods, the inhibitor of TGF-beta activity is a selective inhibitor of a TGF-beta receptor.

In an embodiment, the TGF-beta activity is inhibited via RNAi. In an embodiment, TGF-beta activity is not inhibited via RNAi. In an embodiment, the TGF-beta activity is inhibited through RNAi inhibition of TGF-beta expression, for example by administering an siRNA or an shRNA. An siRNA (small interfering RNA) as used in the methods or compositions described herein comprises a portion which is complementary to an mRNA sequence encoding a mammalian TGF-beta1, TGF-beta2 or TGF-beta3, e.g. in a non-limiting example, GenBank: M60316.1, and the siRNA is effective to inhibit expression of mammalian TGF-beta. In an embodiment, the siRNA comprises a double-stranded portion (duplex). In an embodiment, the siRNA is 20-25 nucleotides in length. In an embodiment the siRNA comprises a 19-21 core RNA duplex with a one or 2 nucleotide 3′ overhang on, independently, either one or both strands. The siRNA can be 5′ phosphorylated or not and may be modified with any of the known modifications in the art to improve efficacy and/or resistance to nuclease degradation. In an embodiment the siRNA can be administered such that it is transfected into one or more cells.

In one embodiment, a siRNA of the invention comprises a double-stranded RNA wherein one strand of the double-stranded RNA is 80, 85, 90, 95 or 100% complementary to a portion of an RNA transcript of a gene encoding mammalian TGF-beta. In another embodiment, a siRNA of the invention comprises a double-stranded RNA wherein one strand of the RNA comprises a portion having a sequence the same as a portion of 18-25 consecutive nucleotides of an RNA transcript of a gene encoding mammalian TGF-beta. In yet another embodiment, a siRNA of the invention comprises a double-stranded RNA wherein both strands of RNA are connected by a non-nucleotide linker. Alternately, a siRNA of the invention comprises a double-stranded RNA wherein both strands of RNA are connected by a nucleotide linker, such as a loop or stem loop structure.

In one embodiment, a single strand component of a siRNA of the invention is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length. In another embodiment, a siRNA of the invention is 40, 41, 42, 43, 44, 45, 46, 47, 48, 49, 50, 51, or 52 nucleotides in length. In yet another embodiment, a siRNA of the invention is 46 nucleotides in length.

In another embodiment, an siRNA of the invention comprises at least one 2′-sugar modification. In another embodiment, an siRNA of the invention comprises at least one nucleic acid base modification. In another embodiment, an siRNA of the invention comprises at least one phosphate backbone modification.

In one embodiment, RNAi inhibition of TGF-beta activity is effected by a short hairpin RNA (“shRNA”). The shRNA can be introduced into the cell by transduction with a vector. In an embodiment, the vector is a lentiviral vector. In an embodiment, the vector comprises a promoter. In an embodiment, the promoter is a U6 or H1 promoter. In an embodiment the shRNA encoded by the vector is a first nucleotide sequence ranging from 19-29 nucleotides complementary to the target gene, in the present case a gene encoding TGF-beta. In an embodiment the shRNA encoded by the vector also comprises a short spacer of 4-15 nucleotides (a loop, which does not hybridize) and a 19-29 nucleotide sequence that is a reverse complement of the first nucleotide sequence. In an embodiment the siRNA resulting from intracellular processing of the shRNA has overhangs of 1 or 2 nucleotides. In an embodiment the siRNA resulting from intracellular processing of the shRNA overhangs has two 3′ overhangs. In an embodiment the overhangs are UU. In an embodiment of the methods and of the compositions herein, the mammalian TGF-BETA is a human TGF-beta.

In an embodiment of the methods, the TGF-beta activity being inhibited is TGF-beta1 activity, TGF-beta2 activity, and/or TGF-beta3 activity. In an embodiment of the methods, the TGF-beta activity is TGF-beta1 activity. In an embodiment of the methods, the TGF-beta activity is TGF-beta2 activity. In an embodiment of the methods, the TGF-beta activity is TGF-beta3 activity.

In an embodiment the TGF-beta, as variously described herein, is a human TGF-beta.

In an embodiment of the methods, the administration of the amount of an inhibitor of TGF-beta activity does not significantly decrease systemic circulation TGF-beta levels in the subject. In embodiments of the methods, the administration of the amount of an inhibitor of TGF-beta activity does not change systemic circulation TGF-beta levels in the subject by more than 0.5%, or by more than 1%. Systemic circulation TGF-beta levels in the subject can be determined by any method for such known in the art.

In an embodiment of the methods, the subject is clinically obese. In an embodiment of the methods, the subject is not clinically obese. In an embodiment of the methods, the subject's age is 40 years or older. In a preferred embodiment, the subject is mammalian. In a most preferred embodiment, the subject is a human.

Also provided is an assay for identifying a treatment for type-2 diabetes comprising contacting a TGF-beta with a small organic molecule and determining if the small organic molecule inhibits activity of the TGF-beta as compared to a non-binding placebo, and positively identifying a small organic molecule which does inhibit activity of the TGF-beta as compared to a non-binding placebo as a treatment for type-2 diabetes. In an embodiment, the assay further comprises determining if the small organic molecule is capable of crossing a mammalian blood-brain barrier, wherein if the small organic molecule is not capable of crossing a mammalian blood-brain barrier it is identified as not a suitable treatment for type-2 diabetes.

Also provided is an assay for identifying a treatment for type-2 diabetes comprising contacting a TGF-beta receptor with a small organic molecule and determining if the small organic molecule inhibits activity of the TGF-beta receptor in the presence of TGF-beta as compared to a non-binding placebo, and positively identifying a small organic molecule which does inhibit activity of the TGF-beta receptor as compared to a non-binding placebo as a treatment for type-2 diabetes. In an embodiment, the assay further comprises determining if the small organic molecule is capable of crossing a mammalian blood-brain barrier, wherein if the small organic molecule is not capable of crossing a mammalian blood-brain barrier it is identified as not a suitable treatment for type-2 diabetes.

An inhibitor of TGF-beta activity is provided for treating type-2 diabetes in a subject. In an embodiment, the inhibitor of TGF-beta activity is formulated for entry into the central nervous system (CNS) of a subject. In an embodiment, the inhibitor of TGF-beta activity is formulated for administration directly into the central nervous system (CNS) of a subject. In an embodiment, the inhibitor of TGF-beta activity is formulated for systemic administration to a subject so as to cross the blood-brain barrier of the subject into the central nervous system (CNS) of the subject in a therapeutic amount.

An inhibitor of TGF-beta activity is provided for reducing development of type-2 diabetes in a subject. In an embodiment, the inhibitor of TGF-beta activity is formulated for entry into the central nervous system (CNS) of a subject. In an embodiment, the inhibitor of TGF-beta activity is formulated for administration directly into the central nervous system (CNS) of a subject. In an embodiment, the inhibitor of TGF-beta activity is formulated for systemic administration to a subject so as to cross the blood-brain barrier of the subject into the central nervous system (CNS) of the subject in a therapeutic amount.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

Hypothalamic TGF-β excess in pro-T2D etiological conditions: In etiology, obesity and aging are known as two important physiological conditions that lead to T2D development. In recent research (12-27), it has been demonstrated that obesity and aging are both associated with hypothalamic inflammation, which is induced by a mild, chronic activation of inflammatory NF-κB signaling. In this study, HFD-fed vs. chow-fed mice were comparatively analyzed, as were middle-aged vs. young mice, and caloric restriction (CR) vs. ad libitum-fed mice, for the hypothalamic expression levels of genes which are closely associated with inflammation but are not strongly pro-inflammatory. With interest, it was observed that hypothalamic TGF-β1 levels were changed across these conditions. As shown in Table 1, Tgfb1 mRNA levels increased in HFD-fed mice as well as aged mice compared to their controls, and these increases were prevented by short-term CR, an approach which exerts effects in counteracting against not only obesity but aging.

TABLE 1
Hypothalamic expression profiles of C57BL/6 mice at young vs. old
ages or under conditions of HFD vs. chow, and AL vs. CR.

| | HFD/Chow | | | Old/Young | | | CR/AL | | |
|---|---|---|---|---|---|---|---|---|---|
| Genes | Fold | SEM | p | Fold | SEM | p | Fold | SEM | p |
| IL11 | 0.52 | 0.21 | 0.25 | 1.11 | 0.28 | 0.72 | 1.18 | 0.50 | 0.57 |
| IL13 | 2.71 | 1.71 | 0.40 | 0.82 | 0.12 | 0.89 | 1.62 | 1.17 | 0.25 |
| IL15 | 1.48 | 0.18 | 0.04* | 1.93 | 0.00 | 0.04* | 0.61 | 0.35 | 0.12 |
| TGF-β1 | 1.92 | 0.29 | 0.02* | 2.41 | 0.29 | 0.0002*** | 0.33 | 0.06 | 0.0004*** |
| PAI1 | 1.09 | 0.07 | 0.80 | 1.08 | 0.09 | 0.46 | 1.02 | 0.26 | 0.18 |
| ICAM1 | 1.42 | 0.28 | 0.36 | 1.02 | 0.09 | 0.55 | 1.89 | 1.01 | 0.10 |
| CX3CL1 | 0.95 | 0.08 | 0.64 | 1.02 | 0.05 | 0.76 | 2.17 | 0.67 | 0.006** |
| CXCL10 | 2.71 | 0.14 | 0.04* | 2.15 | 0.32 | 0.02* | 0.93 | 0.81 | 0.90 |
| CRP | 1.43 | 0.29 | 0.21 | 0.88 | 0.14 | 0.02* | 2.85 | 1.03 | 0.02* |
| IL13-R | 0.92 | 0.04 | 0.19 | 1.44 | 0.06 | 0.004** | 1.91 | 0.48 | 0.001** |
| CNTF-R | 0.87 | 0.06 | 0.25 | 0.74 | 0.02 | 0.003** | 1.18 | 0.48 | 0.57 |
| CT-1Rα | 0.99 | 0.06 | 0.88 | 0.89 | 0.06 | 0.49 | 1.16 | 0.33 | 0.37 |
| OMSR | 1.17 | 0.05 | 0.29 | 1.12 | 0.06 | 0.11 | 2.08 | 0.55 | 0.003** |
| GP130 | 0.90 | 0.08 | 0.21 | 0.96 | 0.02 | 0.48 | 1.00 | 0.14 | 0.99 |
| LIF-R | 0.84 | 0.09 | 0.21 | 0.93 | 0.04 | 0.48 | 1.50 | 0.46 | 0.03* |
| CSF1-R | 0.83 | 0.05 | 0.04* | 1.07 | 0.07 | 0.52 | 1.06 | 0.37 | 0.77 |

Quantitative RT-PCR data represent fold increase over controls.
HFD: high-fat diet,
CR: caloric restriction,
AL: ad libitum feeding.
Statistical analyses:
*p <0.05,
**p <10⁻²,
***p <10⁻³,
n = 6-8 mice/group.
ND, non-detectable.

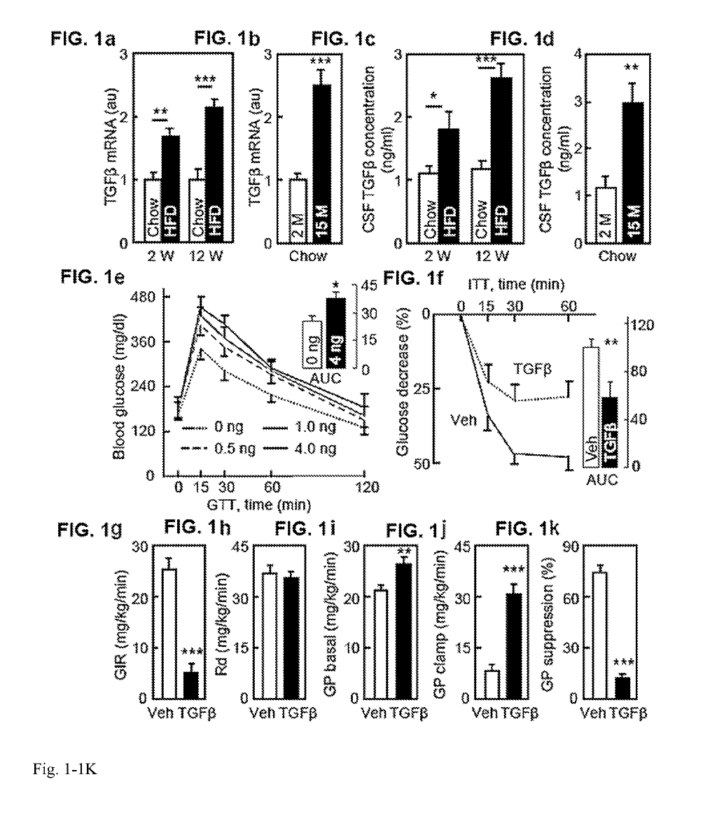

Additional time-course points of HFD feeding or aging were further examined, and it was found that short-term HFD feeding or early-stage aging was both sufficient to increase hypothalamic Tgfb1 mRNA levels (FIG. 1a, b). These mRNA changes indeed led to increased protein levels, as TGF-β contents in the cerebrospinal fluid of HFD-fed mice and aged mice were both higher than matched controls (FIG. 1c, d). In addition to TGF-β1, which is the predominant and most important TGF-β isoform (29), TGF-β2 and 3 were analyzed, and it was found that the hypothalamic mRNA levels of these two isoforms also increased in HFD-fed mice or aged mice. In view of this information, it was decided to study if brain TGF-β excess might have effects on metabolic physiology in mouse models.

Brain TGF-β excess impairs glucose tolerance: To study the metabolic effects of brain TGF-β excess, a pharmacological approach was first used by which TGF-β was delivered into the hypothalamic third ventricle of normal C57BL/6 mice via pre-implanted cannula. To optimize the dosage, different doses of TGF-β1 (0, 0.5, 1.0, and 4.0 ng) were injected, and TGF-β1 concentrations measured in the CSF at various time points post-injection. Data showed that the peak increase of CSF TGF-β1 concentration was seen at 15 min post-injection in a dose-dependent manner, and at 60 min post-injection, TGF-β1 in the CSF declined to the concentrations which were roughly 2-fold higher than the basal concentration. None of these doses significantly increased the blood TGF-β1 concentration. Based on this condition, overnight-fasted mice were given intra-third ventricle injections of TGF-β1 in these doses, one injection at night after food was removed, and the second injection in the following morning at 4 hours prior to glucose tolerance test (GTT). Data revealed that TGF-β1 treatment at each dose led to glucose intolerance, and the effect from 4 ng TGF-β was slightly strongest (FIG. 1e). Following this observation, it was studied whether impaired glucose tolerance in these mice was a result of insulin resistance, and through insulin tolerance test (ITT), data were obtained showing that the sensitivity of insulin in lowing blood glucose was significantly dampened in TGF-β1-injected mice (FIG. 1f). In contrast to these effects on blood glucose, the same protocol of TGF-β1 treatment did not affect food intake or body weight for 24 hours after injection. In sum, a pharmacological induction of brain TGF-β excess can acutely cause pre-diabetic changes including glucose intolerance and insulin resistance.

Brain TGF-β excess increases hepatic glucose production: In physiology, blood glucose levels are balanced by glucose uptake in metabolic tissues (such as muscles) and glucose production in the liver. A hyperinsulinemic-euglycemic clamp was employed to analyze if any of these metabolic processes was relevant to the pro-diabetic effect of brain TGF-β excess. In the experiment, normal C57BL/6 mice were pre-implanted with a catheter into the jugular vein and also a cannula into the hypothalamic third ventricle. Following surgical recovery, overnight-fasted mice were injected with TGF-β1 (4 ng) using the same protocol as described in FIG. 1e, and were subjected to the clamp procedure. During the clamp period, blood glucose concentrations were maintained at approximate 120-130 mg/dl, with steady insulin infusion (4 mU kg⁻¹ min⁻¹) together with various rates of 20% glucose infusion to maintain euglycemia. Glucose infusion rates in TGF-β1-injected mice were significantly lower than vehicle-injected mice since the first 20 min of clamp. While the basal blood insulin concentrations in TGF-β-injected mice were higher which indicated insulin resistance, constant insulin infusion in the clamp elevated the blood insulin concentrations of these mice and controls to similar levels. The calculated data revealed that as a response to similarly increased insulin concentrations, glucose infusion rate in TGF-(β1-injected mice was on average 80% lower than vehicle-injected mice (FIG. 1g). This change was consistent with the data in GTT, both showing that these mice were glucose intolerant (FIG. 1e). The rates of glucose disposal were, however, similar between TGF-β1 and vehicle-injected mice (FIG. 1h), suggesting that tissue glucose uptake was not significantly altered by brain TGF-β. In contrast, even under the basal condition, hepatic glucose production in TGF-β-treated mice was higher compared to control mice (FIG. 1i), and during clamp, while insulin infusion suppressed hepatic glucose production in control mice by ˜75%, it failed to do so in TGF-β1-treated mice (FIG. 1j). Blood glucagon concentrations were similar between TGF-β1-treated mice and controls. Therefore, the brain-liver axis mediates the pro-diabetic effect of brain TGF-β excess.

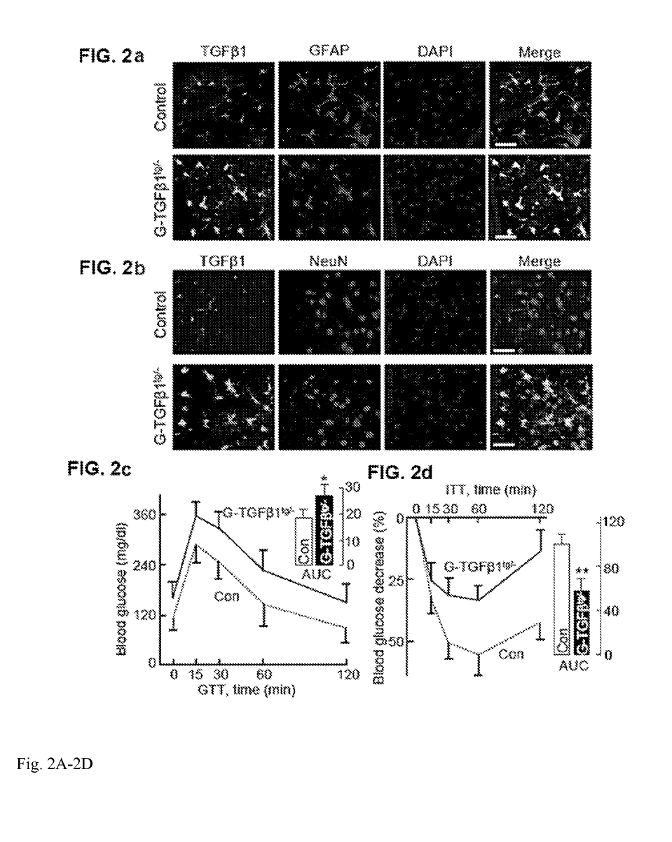

Genetic model of astrocytic TGF-β excess is pro-diabetic: Following the pharmacological studies, a genetic mouse model was developed with brain TGF-β1 excess using transgenic mouse line containing CMV-flox-stop-flox-Tgfb1 in the genome (30). As established (30), the floxed fragment in this transgenic line prevents expression of transgenic TGF-β1, while introduction of Cre leads to the removal of the floxed fragment and therefore TGF-β1 overexpression in Cre-positive cells. In the literature, it has been shown that brain TGF-β is produced mainly from astrocytes (31), and indeed, TGF-β1 was detected predominantly in astrocytes rather than neurons in C57BL/6 mice. Hence, astrocyte-specific TGF-β1 transgenic mice were generated by breeding CMV-flox-stop-flox-Tgfb1 with astrocyte-specific (GFAP-Cre) mice, and the compound mice obtained termed “GFAP-Tgfb1tg/−” mice and littermate control “Tgfb1tg/−” mice. Hypothalamic immunostaining images demonstrated that while TGF-β1 was detected in the astrocytes of control mice, the expression levels were specifically increased in the astrocytes of GFAP-Tgfb1tg/− mice (FIG. 2a, b). GFAP-Tgfb1tg/− and littermate control mice were maintained under chow feeding, and it was confirmed that they had normal development as well as normal food intake and body weight (FIG. 2c, d). In contrast, compared to the normal glucose profile in control mice, chow-fed GFAP-Tgfb1tg/− mice were glucose intolerant (FIG. 2e), and even fasting blood glucose levels of these mice tended to be higher. ITT revealed that GFAP-Tgfb1tg/− mice were severely insulin intolerant (FIG. 2f). Taken together, GFAP-Tgfb1tg/− mice developed glucose intolerance and insulin resistance independently of body weight change.

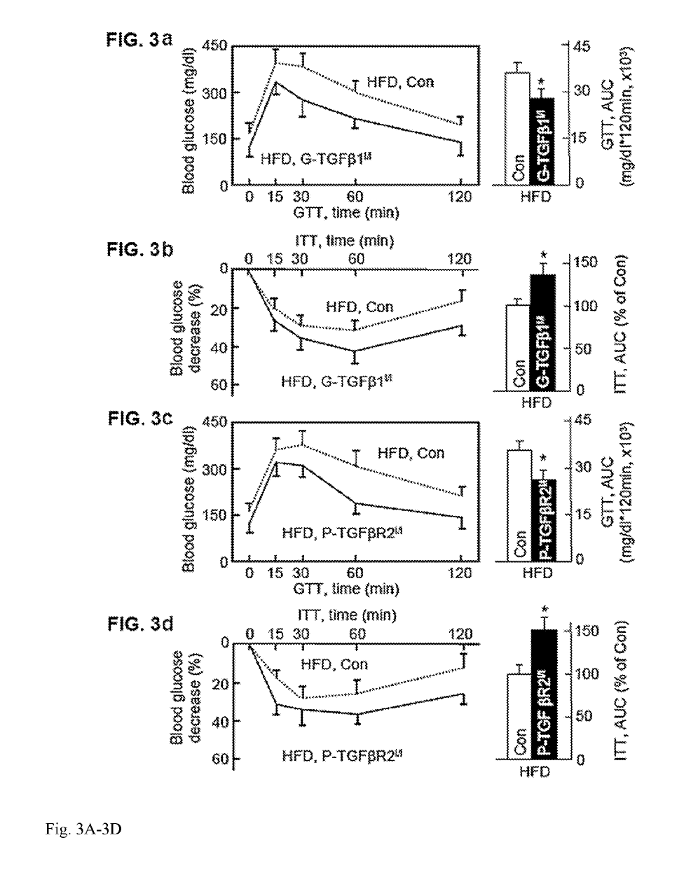

Genetic inhibition of astrocytic TGF-β is anti-diabetic: In parallel with the TGF-β1 gain-of-function model, how an inhibition of brain TGF-β could affect blood glucose levels in physiology or disease was studied. Because astrocytes are important for brain TGF-β excess and the consequent pro-diabetic outcome (FIG. 2), astrocytes were further targeted by breeding Tgfb1lox/lox mice with GFAP-Cre mice, resulting in astrocyte-specific Tgfb1 knockout (GFAP-Tgfb1lox/lox) mice and littermate control Tgfb1 lox/lox mice. When maintained under chow feeding, it was found that GFAP-Tgfb1lox/lox mice and control mice were comparable in terms of development, food intake, body weight, glucose tolerance and insulin tolerance. In the meanwhile, HFD feeding was employed to induce glucose and insulin intolerance and test how these disorders were affected by astrocytic TGF-β inhibition. To better appreciate a primary, obesity-dissociable pro-diabetic mechanism of brain TGF-β, HFD feeding was used for a relative short duration (3 weeks), because while this dietary treatment is sufficient to induce glucose and insulin intolerance, it helpfully addressed if a pro-diabetic brain mechanism could occur in early-stage rather than late-stage obesity development in which complex peripheral mechanisms are pronounced. Following 3-week HFD feeding, it was confirmed that control mice developed impairments in glucose and insulin resistance; in contrast, GFAP-Tgfb1lox/lox mice were found resistant to both of these changes (FIG. 3a, b). Under this 3-week HFD regime, food intake and body weight levels in GFAP-Tgfb1lox/lox mice and control mice were, however, still comparable. Hence, supported by astrocyte-specific TGF-β1 loss-of-function as well as gain-of-function models, it appears astrocytes are important for the pro-diabetic effect of brain TGF-β excess.

POMC neurons direct the pro-T2D effect of TGF-β excess: It was subsequently studied if hypothalamic neurons are critical for the pro-diabetic effects of brain TGF-β excess. Research has revealed that TGF-β signaling increased in the hypothalamus of aged mice, and TGF-β was further shown to inhibit pro-opiomelanorcortin (POMC) peptide in the hypothalamus (32,33). It was also found that Pomc mRNA levels in the hypothalamus of TGF-β1-injected mice were lower compared to the controls. It was studied if POMC neurons could be crucial for the pro-diabetic effect of brain TGF-β excess. To do so, TGF-β receptor-2 (TGFβR2) was targeted, given that TGFβR2 is required for TGF-β signaling, and experimentally TGFβR2 knockout has been shown to inhibit TGF-β signaling (32). To carry out this study, a genetic mouse model was generated with Tgfbr2 knockout specifically in POMC neurons by crossing Tgfbr2lox/lox mice with POMC-Cre mice. As a result, compound offspring POMC-Tgfbr2lox/lox mice were obtained and littermate control Tgfbr2lox/lox mice. These mice were maintained on normal chow feeding or 3-week HFD feeding. Data showed that chow-fed POMC-Tgfbr2lox/lox mice and controls demonstrated normal levels in food intake, body weight and glucose and insulin tolerance. On the other hand, under the condition of 3-week HFD feeding, while food intake and body weight of these mice were still comparable, HFD-induced glucose and insulin intolerance were both markedly lessened in POMC-Tgfbr2lox/lox mice (FIG. 3c, d). Taken together, the action of TGF-β in POMC neurons is important for the pro-diabetic effect of brain TGF-β excess. POMC-Cre can target other types of neurons during the developmental stage (34), thus, the pro-diabetic mechanism of TGF-β may involve additional hypothalamic or brain neurons.

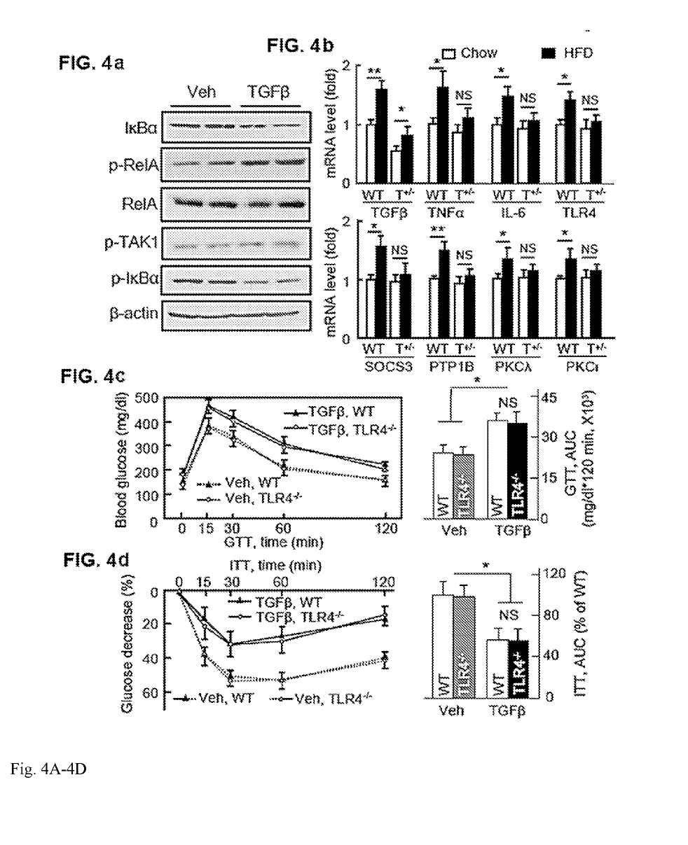

Induction of hypothalamic NF-κB activation by TGF-β: TGF-β is often appreciated for anti-inflammatory feature in immune response, but, depending on physiological context and particularly in pathological conditions, TGF-β can be inflammatory (28)—although it is atypical and many details are still unclear. Here, because NF-κB has been known to atypically mediate hypothalamic inflammation in obesity or aging (12-14,18-20,23-27), it was analyzed if NF-κB signaling components were different in the hypothalamus of mice with third-ventricle injection of TGF-β vs. vehicle. Data revealed that while many of these components had similar protein levels between two groups, TGF-β1 treatment led to a significant reduction in IκBα protein levels (FIG. 4a). Because IκBα is the canonical and specific inhibitor of NF-κB, this result suggested that hypothalamic NF-κB was activated in TGF-β1-treated mice, and the subsequent experiments confirmed this hypothesis. First, since IκBα loss is the specific step that immediately liberates cytoplasmic NF-κB for nuclear translocation, and NF-κB subunit RelA in the nucleus undergoes phosphorylation, RelA phosphorylation was measured in mice injected with TGF-β1 or vehicle. The results revealed that TGF-β1 injection significantly increased hypothalamic RelA phosphorylation (FIG. 4a). Second, it was evaluated if TGF-β1-triggered NF-κB activation could be involved in HFD-induced hypothalamic inflammation. By employing heterogeneous Tgfb1 knockout (Tgfb1 +/−) mice, whether haplodeficiency of Tgfb1 in this mouse model could affect the induction of hypothalamic inflammation by HFD feeding was examined To do so, adult Tgfb1+/− mice and littermate WT controls were subjected to HFD feeding for three weeks, and chow feeding was included to provide as dietary control. Indeed, hypothalamic Tgfb1 mRNA in chow-fed Tgfb1+/− mice dropped by ˜50% compared to chow-fed WT (FIG. 4b), which was consistent with the literature (35). Under 3-week HFD feeding, hypothalamic Tgfb1 mRNA increased significantly in WT mice, but to a much lesser extent in HFD-fed Tgfb1+/− mice (FIG. 4b). Using these hypothalamic samples, we examined mRNA levels of a list of inflammation-related molecules including TNFα, IL-6, SOCS3, TLR4, PTP1B, PKCλ and PKCι. Results demonstrated that 3-week HFD feeding increased hypothalamic mRNA levels of these genes in WT mice but barely in Tgfb1+/− knockout mice (FIG. 4b). Therefore, brain TGF-β excess plays a role in inducing diet-induced hypothalamic inflammation.

Kinase-independent, hypothalamic NF-κB activation by TGF-β: While TGF-β1 clearly led to activation of hypothalamic NF-κB, it was noted that it did not change the phosphorylated levels of IκBα when normalized by IκBα protein levels (FIG. 4a). Thus, while kinase (e.g., IKK)-induced IκBα phosphorylation is a crucial, rapid signaling reaction in classical inflammation, this process is not primarily critical in TGF-β1-induced hypothalamic inflammation.

It was also examined whether TAK1, a kinase which can mediate TGF-β-induced NF-κB activation in some immune cells, was relevant, but data revealed that TGF-β1 did not lead to hypothalamic TAK1 phosphorylation (FIG. 4a). All these observations suggest that TGF-β1 modulates IκBα levels in a manner which is independent of upstream kinase signaling, leading to atypical activation of hypothalamic NF-κB. To further assess this point, it was asked if the effects of brain TGF-β excess could be impaired by ablation of a signaling component, such as toll-like receptor-4 (TLR4) or myeloid differentiation primary response gene 88 (MyD88), because they employ kinase signaling to induce IκBα phosphorylation and thus NF-κB activation. Using a genetic model, T1r4 knockout (T1r4−/−) mice, TGF-β1 or vehicle was delivered into the hypothalamic third ventricle of these mice and littermate WT mice via pre-implanted cannula. Vehicle-injected T1r4−/− mice and WT mice were both normal in glucose and insulin tolerance tests (FIG. 4a, b). Of note, TGF-β1 treatment led to similar extents of glucose intolerance and insulin resistance in T1r4−/− mice and WT mice (FIG. 4c, d). Also, using Myd88 knockout mice, it was observed that the lack of Myd88 did not lead to a significant reduction in glucose or insulin intolerance following hypothalamic third ventricle TGF-β1 delivery (data not shown). Altogether, brain TGF-β excess may use a mechanism that directly targets IκBα rather than upstream kinase signaling to activate hypothalamic NF-κB.

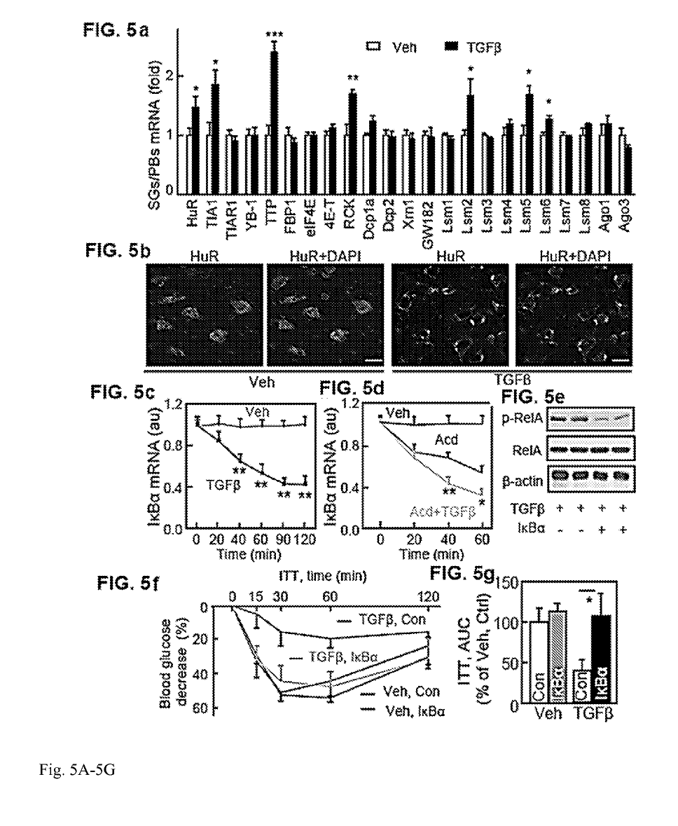

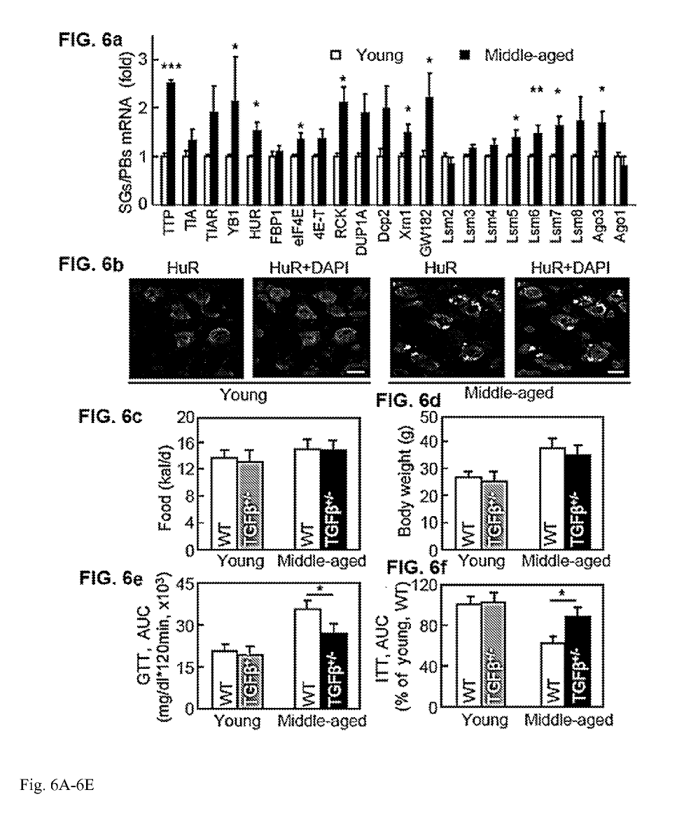

Induction of hypothalamic RNA SGs/PBs by TGF-β or HFD: In exploring how TGF-β could activate hypothalamic NF-κB without requiring pro-inflammatory kinase signaling, attention was directed to a possible role from mRNA regulation. In coping with inflammatory stress, eukaryotic cells can develop a process known as RNA stress response which is characterized by RNA stress granules (SGs) and processing bodies (PBs) (36-38). In this reaction, messenger ribonuclear protein (mRNP) byproducts exit from polysomes and form RNA SGs at discrete cytoplasmic foci. RNA SGs primarily consist of poly(A)+mRNAs-containing 48S pre-initiation complexes, small ribosomal subunits, mRNA decay factor tristetraprolin (TTP), translation initiation factors such as eukaryotic translation initiation factor-4E (eIF4E), eIF4G, eIF4A, eIF4B, poly(A)-binding protein (PABP), and RNA helicases (36-38). RNA stress response can lead to the export of mRNPs into the PBs, a complex which harbors an array of mRNA decay machineries that act to dispose mRNAs from SGs or polysomes (36-38). PBs contain nontranslating mRNAs, translation repressors, mRNA decay machineries (including 5′-3′ mRNA decay system, nonsense-mediated decay pathway, and RNA-induced silencing complex), and mRNA decay factors such as TTP, eIF4E, DEAD box RNA helicase family member p54/RCK, cAMP response element-binding transcription factor (CPEB), B-related factor 1/RNA polymerase III transcription initiation factor IIIB subunit (BRF1), eukaryotic translation initiation factor 4E transporter (4-ET), and RNA-binding protein Smaug (36-38). In this context, the expression levels of these RNA SGs/PBs genes were analyzed in the mouse models, and it was found that many of them were increased in the hypothalamus of C57BL/6 mice with 3-month HFD feeding. Notably, hypothalamic increases of these genes were similarly induced by an injection of TGF-β1 into the third ventricle of normal mice (FIG. 5a). In line with these observations, we examined if the morphology of RNA SGs could be detected in the hypothalamus of these mice. We performed immunostaining of HuR, a molecular component of RNA SGs, and found that HuR-containing aggregates were present in the perinuclear regions of hypothalamic cells in TGF-β1-injected mice but barely in control mice (FIG. 5b). Consistently, these morphological changes were found in the hypothalamus of HFD-fed mice but almost not chow-fed mice (supplementary FIG. 6b). These data suggest that RNA stress response could be causally important for the induction of obesity-associated hypothalamic inflammation.

TGF-β degrades IκBα mRNA to atypically activate NF-κB: RNA SGs/PBs have the function to degrade mRNAs by targeting the AU-rich element (AUE) at the 3′ untranslated region (UTR) (36-38). Analysis of gene sequences showed that AUE is conserved in IκBα mRNA across species. This information led to the suspicion that TGF-β could work to degrade IκBα mRNA. Using both hypothalamic GT1-7 cells (FIG. 5c) and HEK 293 cells (data not shown), experiments revealed that IκBα mRNA levels in these cells notably decreased following TGF-β1 treatment. When these cells were added with transcription inhibitor actinomycin D, IκBα mRNA decay further accelerated (FIG. 5d). These results indicated that the turnover of IκBα mRNA is fast, and TGF-β has a strong effect in promoting IκBα mRNA decay. Consistent with in vitro data, an intra-third ventricle injection of TGF-β1 was sufficient to decrease hypothalamic IκBα mRNA levels (FIG. 5e), and it was predicted that this mRNA change led to hypothalamic IκBα protein loss in TGF-β1-injected mice (FIG. 4a). To test if TGF-β induced IκBα mRNA decay is accountable for the glucose metabolism disorder, an experiment was designed to study if the pro-diabetic effect of brain TGF-β excess could be reversed through directly increasing hypothalamic IκBα mRNA. Using an approach which was established previously (23), a lentiviral system was employed to deliver exogenous Iκbα mRNA into the mediobasal hypothalamus of chow-fed C57BL/6 mice, and indeed, this lentivirus-delivered Iκbα mRNA prevented TGF-β1 injection from activating hypothalamic NF-κB (FIG. 5f). Metabolic analysis confirmed that this treatment attenuated the effect of TGF-β1 from impairing insulin-dependent glucose control (FIG. 5g). Altogether, the findings suggest that a hypothalamic process consisting of TGF-β excess, RNA stress response and IκBα mRNA decay mediates the pro-diabetic mechanism of the brain in the condition of dietary obesity.

Hypothalamic TGF-β and RNA SGs/PBs link aging to glucose intolerance: Finally, it was studied if the pro-diabetic role of hypothalamic TGF-β-directed RNA SGs/PBs is also important for aging-related glucose and insulin disorders. In Table 1, it is shown that hypothalamic TGF-β1 mRNA levels increased in aged mice but were decreased by CR. In this context, expression levels of RNA SBs/PBs components were analyzed, and it is found that many of these molecules were upregulated in the hypothalamus of aged mice compared to young mice (FIG. 6a). Using immunostaining, it was confirmed that RNA SGs were present in the hypothalamus of old mice but barely in young mice (FIG. 6b). These aging-associated changes significantly overlap with those induced by HFD feeding, implicating that dietary obesity and aging have a common abnormality dictated by RNA stress response-triggered inflammation. In this context, Tgfb1+/− mice were used to study if the partial inhibition of TGF-β1 in this model could protect against aging-induced glucose and insulin intolerance. In this experiment, Tgfb1+/− mice and WT controls were maintained under chow feeding and studied for metabolic physiology in young vs. middle-aged conditions. The follow-up showed that chow-fed Tgfb1+/− mice and WT controls had similar food intake and body weight (FIG. 6c, d). At young ages, Tgfb1+/− mice and WT had similar glucose levels in GTT and ITT (FIG. 6e, f). Glucose and insulin tolerance were both impaired in middle-aged WT mice compared to young WT mice; in contrast, middle-aged Tgfb+/− mice showed significant improvements of glucose tolerance in GTT and insulin tolerance in ITT (FIG. 6e, f). Taken together, TGF-β excess and inflammatory RNA metabolism represent two critical factors located in the crossroad of translating not only obesity but also aging into pro-diabetic complications.

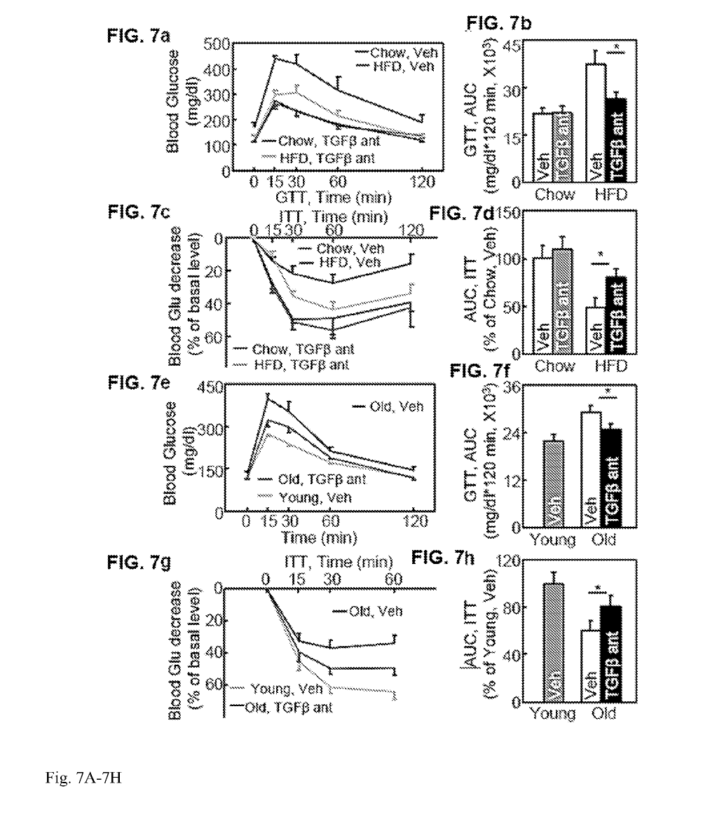

Central TGFβ blockade reverses glucose disorder under obesity or aging: It was studied whether TGFβ blockade could be applied to the CNS in order to reverse glucose and insulin disorders under disease conditions. To address this question, a mouse model was employed with HFD-induced obesity and therefore glucose and insulin intolerance. In this study, C57BL/6 mice were maintained under a HFD vs. a chow for 3 months. Subsequently, TGFβ antagonist SB431542 vs. vehicle was administrated to overnight-fasted mice through pre-implanted cannula. It was found that a single injection of SB431542 in the third ventricle significantly reduced glucose intolerance (FIG. 7a&b) and insulin intolerance (FIG. 7c&d). These therapeutic effects were demonstrated during 2-hour treatment duration, which were independent of feeding or body weight. In parallel, using aging paradigm, it was further asked if TGFβ blockade could be used to intervene with glucose and insulin disorders independently of HFD feeding. To address this question, a similar procedure was applied to young vs. old mice; both mice were maintained on a normal chow since weaning. As shown in FIG. 7e-h, aging was associated with impairment of glucose tolerance and insulin tolerance in control group. However, hypothalamic third-ventricle injection of SB431542 led to significant reductions in glucose and insulin intolerance (FIG. 7e-h). Altogether, central inhibition of TGFβ through a pharmacological approach acutely normalizes the brain function to improve glucose and insulin homeostasis and treat type-2 diabetes.

### Discussion

Glucose intolerance by TGF-β is potentially adaptive but chronically pro-diabetic. Based on epidemiological and clinical evidences, hyperglycemia and glucose intolerance are frequently found in brain diseases such as Alzheimer's disease (39-41). Recently, manipulations of the CNS or the hypothalamus were found to change hepatic glucose production in experimental models (5-9), but it remains unexplored whether the brain could mediate diabetic development. Here, it is found that obesity and aging are both associated with overproduction of TGF-β in the brain, and our pharmacological and genetic models consistently revealed that excess of TGF-β in the brain leads to glucose and insulin intolerance in a manner which is dissociable from obesity or aging. The brain-liver axis was found critical for the pro-diabetic effect of brain TGF-β excess, agreeing with the knowledge demonstrating that the hypothalamus has a regulatory role in hepatic glucose production (5-8). Conceptually, the finding was in line with the literature showing that TGF-β excess was not only seen in many brain diseases but also implicated in their pathogenesis (42-46). On the other hand, despite this disease relevance of TGF-β excess, it needs to be borne in mind that TGF-β has biological functions in cell growth, differentiation and transformation, and complete absence of TGF-β is developmentally lethal (47,48). With regards to the CNS, lack of brain TGF-α signaling can affect neurological development or synapse function (32,49,50), indicating that a normal level of brain TGF-β is biologically required and therefore neuroprotective. Against this background, it is possible to consider that increase of TGF-β in brain diseases may represent an adaptive response, and by inducing glucose intolerance, it can increase glucose availability for the brain, since glucose is almost the exclusive fuel for the brain, and an increase in glucose flux can help the brain to cope with stress and damages. However, when such induction of glucose intolerance is chronic, it lowers the threshold of developing diabetes, leading to the T2D-prone condition. Consistent with this idea, systemic TGF-β neutralization in db/db mice or ob/ob mice was shown to have effects in reducing glucose or renal disorders in the context of body weight reduction (51,52). Herein, the findings demonstrate that brain TGF-β excess is pro-diabetic which is independent of body weight, and therefore, appropriate TGF-β suppression can represent a therapeutic basis for diabetic patients with or without obesity.

A mediator of atypical hypothalamic inflammation—the RNA stress response: Classical NF-κB activation is induced by membrane receptor-dependent kinases such as IKK and TAK153. Activation of these kinases rapidly leads to IκBα phosphorylation, ubiquitination and degradation, and subsequently, NF-κB is liberated from binding to IκBα, enters the nucleus and induce gene transcription (53). This paradigm of classical NF-κB activation requires extracellular stimuli, such as pathogens or related molecules which activate TLRs. As a result, activated NF-κB induces gene expression of inflammatory cytokines (e.g., TNF-α and interleukins), which are released to induce subsequent NF-κB activation through cytokine receptor signaling. Recently, obesity and aging were both revealed to be associated with hypothalamic NF-κB activation (12-14,18-20,23-27); however, how hypothalamic NF-κB activation is triggered in these conditions was unclear. Here, it is demonstrated that hypothalamic RNA stress response induces IκBα mRNA decay to initiate NF-κB activation, an intracellular RNA metabolism-driven event which does not rely on receptor signaling. In the literature, it has been documented that the biological function of RNA stress response is to provide an early intracellular defense during which RNA SGs/PBs are formed to degrade ARE-containing mRNAs (36-38). In this study, it was demonstrated that IκBα mRNA has a fast turnover rate and is sensitively subjected to RNA SGs/PBs-mediated mRNA decay. TGF-β excess can trigger this process, despite that it remains to be studied if there are other contributing factor(s). Furthermore, it was found that induction of NF-κB-dependent inflammatory genes by short-term HFD feeding was suppressed by TGF-β1 inhibition, suggesting that TGF-β excess is involved in initiating obesity-related hypothalamic inflammation. In physiology, since hypothalamic NF-κB mediates the pro-diabetic effect of brain TGF-β excess, it provides a strong support to an integrated model that the brain mechanism of T2D involves the activation of hypothalamic NF-κB by many other factors, such as endoplasmic reticulum stress, cytokines and other inflammatory signaling molecules (e.g., JNK). While the predicted pro-diabetic effects of these factors are intertwined with obesity development, here an obesity-independent mechanism was dissected out to support the conclusion that hypothalamic inflammation is primarily involved in diabetic development.

Inflammatory milieu—key for disease relevance of inflammation-related cytokines: TGF-β has often been studied for regulating immune cells, tissue remodeling, wound healing and fibrosis which were frequently recognized anti-inflammatory (28). Here, we demonstrated that brain TGF-β excess atypically activates NF-κB which is pro-inflammatory. In agreement, it has been recently appreciated that TGF-β can support pro-inflammatory functions in the context of other inflammation-related cytokines (28,54,55). Thus, the study here gives an example to indicate that it is simplistic to unconditionally label a cytokine “anti-inflammatory” or “pro-inflammatory” when addressing its physiological relevance. In light of TGF-β, although being counter-inflammatory in molecular signaling, if inflammation is not resolved timely, chronic excess of this cytokine contributes to the inflammatory mechanism of physiological dysfunctions and disease. This is also to say, the inflammatory milieu can determine how an anti-inflammatory cytokine affects physiological functions. For example, it was shown here that brain TGF-β excess in the context of early-stage hypothalamic inflammation increases the body's sensitivity to the development of pro-diabetic glucose disorders, implicating that an “anti-inflammatory” cytokine plays a part in the inflammatory network associated with metabolic disease. In alignment with this appreciation, it is likely that divergent networks of individual pro- and anti-inflammatory factors can formulate hypothalamic inflammation in chronic diseases which involve different metabolic changes (such as obesity vs. cachexia), and inflammatory milieu should be considered in designing approaches to tackle the inflammatory mechanism of a specific metabolic disease.

### Materials and Methods

Animals. Tgfb1^{lox/lox} mice, Tgfbr2^{lox/lox} mice, Tgfb1^{+/-} mice, GFAP-Cre mice, and T1r4^{-/-} mice on C57BL/6 were obtained from Jackson (32,56-59) and continued to be maintained on C57BL/6. CMV-lox-stop-lox-Tgfb1 mice(30) obtained from Jackson were backcrossed into C57BL/6. POMC-Cre mice maintained on C57BL/6 were used in our previous research (20,60). All mice were housed in standard, pathogen-free animal facility with 12 h/12 h light and darkness cycles, and adult male mice were used in experiments of this work. Mice were maintained on normal chow since weaning, and for some experiments involving HFD feeding, a HFD (45% kcal fat, Research Diets, Inc.) was used when mice were two to three months old. Food intake and body weight of mice were measured using a laboratory scale. GTT was performed in mice through intraperitoneal (i.p.) injection of glucose at 2 g/kg body weight. ITT was performed in mice through i.p. injection of human recombinant insulin (Nova Nordisk) at the dose of 0.7 U/kg body weight. Blood glucose levels during GTT and ITT were measured with LifeScan® blood glucose monitoring system. All procedures were approved by the Institutional Animal Care and Use Committee of Albert Einstein College of Medicine.

Brain injection. As we previously described (14,61), using an ultra-precise (10 μm resolution) small animal stereotactic apparatus (David Kopf Instruments), a 26 gauge guide cannula (Plastics One, Inc.) was implanted into third ventricle of anesthetized mice at the midline coordinates of 1.8 mm posterior to bregma and 5.0 mm below the surface of skull. Intra-third ventricular injection was carried out with a 33-gauge internal cannula (Plastics One) connected to a 5-μl Hamilton Syringe. TGF-β1 (Sigma) was dissolved in 1 μl artificial cerebrospinal fluid (aCSF) for injection. Injection of aCSF was used as vehicle control. Pharmacological treatment: Mice were fasted for an overnight period, and received two injections of TGF-β1 vs. vehicle via pre-implanted cannula, one injection at night after food was removed, and the second injection in the following morning at 4 hours prior to a metabolic test such as GTT and ITT. Bilateral intra-MBH viral injections were directed by an ultra-precise stereotactic apparatus at coordinates of 1.5 mm posterior to bregma, 5.8 mm below the surface of skull, and 0.3 mm lateral to midline, as previously described 14. Purified lentiviruses suspended in 0.2 μl aCSF was injected over 10-min period via a 26-gauge guide cannula and a 33-gauge internal injector (Plastics One) connected to a 5-μl Hamilton Syringe and infusion pump (WPI Instruments).

Hyperinsulinemic-euglycemic clamp. Mice that were pre-implanted with cannula in the third ventricle were anesthetized, and a catheter was inserted into the right jugular vein and crossed over from the underneath and out the back of the neck. Following surgical recovery, overnight-fasted mice were injected with TGF-β1 (4 ng) vs. vehicle, once at the beginning of fasting and the other in the following morning at four hours prior to clamp. Conscious mice were then subjected to euglycemic clamp, with the blood glucose concentrations maintained at 120-130 mg/dl for four hours, followed by steady-state human insulin infusion (4 mU kg−1 min−1) together with infusion of 20% glucose at variable rates to maintain euglycemia. During the final 1 hour of the clamp, [3-³H] glucose and 2-deoxy-D-[1-¹⁴C] glucose (PerkinElmer) infusions were used. Blood samples were collected from tail vein. At the end of the procedure, mice were euthanized, and various tissues were removed and quickly frozen in liquid nitrogen. Plasma insulin and glucagon concentrations were analyzed with ELISA kits (Crystal Chem. and R&D Systems).

Lentiviruses and histology. Lentiviral TTP shRNA and matched control vector were purchased from Sigma (MISSION shRNA System). Lentiviral IκBα was cloned by inserting cDNA of dominant-negative IκBα in synapsin promoter-driven lentiviral vector as previously described 14, and replacement of IκBα by GFP cDNA was used as the matched control. Lentiviruses were generated in HEK293T cells and then purified as previously described 14. Brain histology was analyzed using brain sections and immunostaining. Mice under anesthesia were transcardially perfused with 4% PFA and brains were removed, post-fixed in 4% PFA for four hours, and infiltrated with 20%-30% sucrose. 20 μm-thick brain sections were blocked with serum of appropriate species, penetrated with 0.2% Triton-X 100, treated with primary antibodies including mouse anti-GFAP (Millipore, MAB3402, 1:1000), mouse anti-NeuN (Millipore, MAB377, 1:1000), rabbit anti-TGF-β (Abcam, ab53169, 1:200), and mouse anti-HuR (Santa Cruz, sc5261, 1:500), and subsequently reacted with fluorescent secondary antibodies (Invitrogen, 1:1000). Naïve IgGs of appropriate species were used as negative controls. DAPI staining was used to reveal all cells in the section. Images were taken using a confocal microscope.

Protein, mRNA and peptide analyses. Hypothalami were isolated as previously described (14). Tissue lysis, SDS/PAGE and Western blotting were performed as previously described (19). Primary antibodies in Western blots included anti-IκBα (Santa Cruz, #SC847, 1:500), anti-p-TAK1 (Cell Signaling, #4531, 1:1000), anti-p-IκBα (Cell Signaling, #2859, 1:1000), anti-RelA (Cell Signaling, #3039, 1:1000), anti-p-RelA (Cell Signaling, #4764, 1:1000), and anti-β-actin (Cell Signaling, #4967S, 1:1000), and secondary antibodies were HRP-conjugated anti-rabbit or goat antibody (Pierce, 1:2000). Quantification of Western blots was processed with Image J. Total RNA was extracted from hypothalamic tissue using TRIzol® (Invitrogen), and cDNA was synthesized using M-MLV RT System (Promega). Using SYBR® Green PCR Master Mix (Applied Biosystems), expression levels of target genes were analyzed via PCR amplification and quantification. GAPDH or TBP mRNA levels were used for normalization. CSF collection and TGFβ measurement: An anesthetized mouse was placed onto the stereotactic apparatus with the head forming an angle of about 135° with the body, and then a sagittal incision in the neck skin was made inferior to the occiput, followed by penetrating a capillary tube through the dura mater into the cisterna magna to draw the CSF. Serum and CSF TGF-β content were measured using TGF-β ELISA kit (R&D Systems).

Cell culture. GT1-7 cells were cultured as described previously (14). Briefly, GT1-7 cells were maintained in Dulbecco's Modified Eagle's Medium (Invitrogen) with 10% fetal bovine serum (Hyclone) and penicillin/streptomycin (Invitrogen), at 37° C. in a humidified atmosphere containing 5% CO₂. Cells were fasted in serum-free medium for an overnight period, and then were subjected to TGF-β1 treatment at the indicated dose and time course.

Statistics. Two-tailed Student's t test was used for comparisons between two groups, and ANOVA and appropriate post hoc analyses were used for comparisons among more than two groups. Data presented met normal distribution, and statistical tests for each figure were justified appropriate. Sample sizes were chosen with adequate power based on the literature. Data were presented as mean ±SEM. P<0.05 was considered statistically significant.

### REFERENCES

1. Samuel, V. T. & Shulman, G. I. Mechanisms for insulin resistance: common threads and missing links. Cell 148, 852-871 (2012).
2. Gregor, M. F. & Hotamisligil, G. S. Inflammatory mechanisms in obesity. Annu. Rev. Immunol. 29, 415-445 (2011).
3. Muoio, D. M. & Newgard, C. B. Obesity-related derangements in metabolic regulation. Annu. Rev. Biochem. 75, 367-401 (2006).
4. Glass, C. K. & Olefsky, J. M. Inflammation and lipid signaling in the etiology of insulin resistance. Cell Metab 15, 635-645 (2012).
5. Mighiu, P. I. et al. Hypothalamic glucagon signaling inhibits hepatic glucose production. Nat. Med. 19, 766-772 (2013).
6. Berglund, E. D. et al. Direct leptin action on POMC neurons regulates glucose homeostasis and hepatic insulin sensitivity in mice. J. Clin. Invest 122, 1000-1009 (2012).
7. Obici, S., Zhang, B. B., Karkanias, G., & Rossetti, L. Hypothalamic insulin signaling is required for inhibition of glucose production. Nat. Med. 8, 1376-1382 (2002).
8. Lam, T. K. et al. Hypothalamic sensing of circulating fatty acids is required for glucose homeostasis. Nat. Med. 11, 320-327 (2005).
9. Joly-Amado, A. et al. Hypothalamic AgRP-neurons control peripheral substrate utilization and nutrient partitioning. EMBO J. 31, 4276-4288 (2012).
10. Wisse, B. E., Kim, F., & Schwartz, M. W. Physiology. An integrative view of obesity. Science 318, 928-929 (2007).
11. Cone, R. D. Anatomy and regulation of the central melanocortin system. Nat. Neurosci. 8, 571-578 (2005).
12. Cai, D. Neuroinflammation and neurodegeneration in overnutrition-induced diseases 2250. Trends Endocrinol. Metab 24, 40-47 (2013).
13. Cai, D. & Liu, T. Hypothalamic inflammation: a double-edged sword to nutritional diseases. Ann. N. Y. Acad. Sci. 1243, E1-39 (2011).
14. Zhang, X. et al. Hypothalamic IKKbeta/NF-kappaB and ER stress link overnutrition to energy imbalance and obesity. Cell 135, 61-73 (2008).
15. De Souza, C. T. et al. Consumption of a fat-rich diet activates a proinflammatory response and induces insulin resistance in the hypothalamus. Endocrinology 146, 4192-4199 (2005).
16. Kleinridders, A. et al. MyD88 signaling in the CNS is required for development of fatty acid-induced leptin resistance and diet-induced obesity. Cell Metab 10, 249-259 (2009).
17. Horvath, T. L. et al. Synaptic input organization of the melanocortin system predicts diet-induced hypothalamic reactive gliosis and obesity. Proc. Natl. Acad. Sci. U.S.A. 107, 14875-14880 (2010).
18. Meng, Q. & Cai, D. Defective hypothalamic autophagy directs the central pathogenesis of obesity via the IkappaB kinase beta (IKKbeta)/NF-kappaB pathway. J. Biol. Chem. 286, 32324-32332 (2011).
19. Purkayastha, S. et al. Neural dysregulation of peripheral insulin action and blood pressure by brain endoplasmic reticulum stress. Proc. Natl. Acad. Sci. U.S.A. 108, 2939-2944 (2011).
20. Purkayastha, S., Zhang, G., & Cai, D. Uncoupling the mechanisms of obesity and hypertension by targeting hypothalamic IKK-beta and NF-kappaB. Nat. Med. 17, 883-887 (2011).
21. Milanski, M. et al. Inhibition of hypothalamic inflammation reverses diet-induced insulin resistance in the liver. Diabetes 61, 1455-1462 (2012).
22. Thaler, J. P. et al. Obesity is associated with hypothalamic injury in rodents and humans. J. Clin. Invest 122, 153-162 (2012).
23. Zhang, G. et al. Hypothalamic programming of systemic ageing involving IKK-beta, NF-kappaB and GnRH. Nature 497, 211-216 (2013).
24. Purkayastha, S. & Cai, D. Neuroinflammatory basis of metabolic syndrome. Mol. Metab 2, 356-363 (2013).
25. Tang, Y. & Cai, D. Hypothalamic inflammation and GnRH in aging development. Cell Cycle 12, 2711-2712 (2013).
26. Li, J., Tang, Y., & Cai, D. IKKbeta/NF-kappaB disrupts adult hypothalamic neural stem cells to mediate a neurodegenerative mechanism of dietary obesity and pre-diabetes. Nat. Cell Biol. 14, 999-1012 (2012).
27. Li, J. et al. Control of obesity and glucose intolerance via building neural stem cells in the hypothalamus. Mol. Metab 3, 313-324 (2014).
28. Wan, Y. Y. & Flavell, R. A. ‘Yin-Yang’ functions of transforming growth factor-beta and T regulatory cells in immune regulation. Immunol. Rev. 220, 199-213 (2007).
29. Li, M. O. et al. Transforming growth factor-beta regulation of immune responses. Annu. Rev. Immunol. 24, 99-146 (2006).
30. Hall, B. E. et al. Conditional overexpression of TGF-beta1 disrupts mouse salivary gland development and function. Lab Invest 90, 543-555 (2010).
31. Bouret, S. et al. Evidence that TGF beta may directly modulate POMC mRNA expression in the female rat arcuate nucleus. Endocrinology 142, 4055-4065 (2001).
32. Falk, S. et al. Brain area-specific effect of TGF-beta signaling on Wnt-dependent neural stem cell expansion. Cell Stem Cell 2, 472-483 (2008).
33. Beynon, A. L., Thome, J., & Coogan, A. N. Age and time of day influences on the expression of transforming growth factor-beta and phosphorylated SMAD3 in the mouse suprachiasmatic and paraventricular nuclei. Neuroimmunomodulation. 16, 392-399 (2009).
34. Padilla, S. L., Carmody, J. S., & Zeltser, L. M. Pomc-expressing progenitors give rise to antagonistic neuronal populations in hypothalamic feeding circuits. Nat. Med. 16, 403-405 (2010).
35. Scherf, W., Burdach, S., & Hansen, G. Reduced expression of transforming growth factor beta 1 exacerbates pathology in an experimental asthma model. Eur. J. Immunol. 35, 198-206 (2005).
36. Thomas, M. G., Loschi, M., Desbats, M. A., & Boccaccio, G. L. RNA granules: the good, the bad and the ugly. Cell Signal. 23, 324-334 (2011).
37. Buchan, J. R. & Parker, R. Eukaryotic stress granules: the ins and outs of translation. Mol. Cell 36, 932-941 (2009).
38. Anderson, P. & Kedersha, N. Stress granules: the Tao of RNA triage. Trends Biochem. Sci. 33, 141-150 (2008).
39. Dai, Y. & Kamal, M. A. Fighting Alzheimer's Disease and Type 2 Diabetes: Pathological Links and Treatment Strategies. CNS. Neurol. Disord. Drug Targets. (2013).
40. McCrimmon, R. J., Ryan, C. M., & Frier, B. M. Diabetes and cognitive dysfunction. Lancet 379, 2291-2299 (2012).
41. de la Monte, S. M. & Wands, J. R. Alzheimer's disease is type 3 diabetes-evidence reviewed. J. Diabetes Sci. Technol. 2, 1101-1113 (2008).
42. Vargas, D. L. et al. Neuroglial activation and neuroinflammation in the brain of patients with autism. Ann. Neurol. 57, 67-81 (2005).
43. Ilzecka, J., Stelmasiak, Z., & Dobosz, B. Transforming growth factor-Beta 1 (tgf-Beta 1) in patients with amyotrophic lateral sclerosis. Cytokine 20, 239-243 (2002).
44. Tarkowski, E. et al. Increased intrathecal levels of the angiogenic factors VEGF and TGF-beta in Alzheimer's disease and vascular dementia. Neurobiol. Aging 23, 237-243 (2002).
45. Wyss-Coray, T., Borrow, P., Brooker, M. J., & Mucke, L. Astroglial overproduction of TGF-beta 1 enhances inflammatory central nervous system disease in transgenic mice. J. Neuroimmunol. 77, 45-50 (1997).
46. Grammas, P. & Ovase, R. Cerebrovascular transforming growth factor-beta contributes to inflammation in the Alzheimer's disease brain. Am. J. Pathol. 160, 1583-1587 (2002).
47. Bottinger, E. P., Letterio, J. J., & Roberts, A. B. Biology of TGF-beta in knockout and transgenic mouse models. Kidney Int. 51, 1355-1360 (1997).
48. Dunker, N. & Krieglstein, K. Tgfbeta2 −/− Tgfbeta3 −/− double knockout mice display severe midline fusion defects and early embryonic lethality. Anat. Embryol. (Berl) 206, 73-83 (2002).
49. Heupel, K. et al. Loss of transforming growth factor-beta 2 leads to impairment of central synapse function. Neural Dev. 3, 25 (2008).
50. Vogel, T., Ahrens, S., Buttner, N., & Krieglstein, K. Transforming growth factor beta promotes neuronal cell fate of mouse cortical and hippocampal progenitors in vitro and in vivo: identification of Nedd9 as an essential signaling component. Cereb. Cortex 20, 661-671 (2010).
51. Ziyadeh, F. N. et al. Long-term prevention of renal insufficiency, excess matrix gene expression, and glomerular mesangial matrix expansion by treatment with monoclonal antitransforming growth factor-beta antibody in db/db diabetic mice. Proc. Natl. Acad. Sci. U.S.A. 97, 8015-8020 (2000).
52. Yadav, H. et al. Protection from obesity and diabetes by blockade of TGF-beta/Smad3 signaling. Cell Metab 14, 67-79 (2011).
53. Hayden, M. S. & Ghosh, S. Shared principles in NF-kappaB signaling. Cell 132, 344-362 (2008).
54. Veldhoen, M. et al. TGFbeta in the context of an inflammatory cytokine milieu supports de novo differentiation of IL-17-producing T cells. Immunity. 24, 179-189 (2006).
55. Mangan, P. R. et al. Transforming growth factor-beta induces development of the T(H)17 lineage. Nature 441, 231-234 (2006).

1. A method of treating type-2 diabetes in a subject comprising administering to the subject an amount of an inhibitor of TGF-beta activity, in a manner effective to enter the central nervous system (CNS) of a subject, effective to treat type-2 diabetes in a subject.

2. A method of reducing development of type-2 diabetes in a subject comprising administering to the subject an amount of an inhibitor of TGF-beta activity, in a manner effective to enter the central nervous system (CNS) of a subject, effective to reduce development of type-2 diabetes in a subject.

3. The method of claim 1, wherein the inhibitor of TGF-beta activity is administered directly to the CNS of the subject.

4. The method of claim 1, wherein the inhibitor of TGF-beta activity is administered systemically but is able to cross the blood-brain barrier into the CNS of the subject.

5. The method of claim 1, wherein the inhibitor of TGF-beta activity is administered encapsulated in a liposome.

6. The method of claim 5, wherein the liposome is glutathione-coated.

7. The method of claim 1, wherein the inhibitor of TGF-beta activity comprises an isolated antibody, a fragment of such an antibody, or a synthetic fusion protein comprising a soluble portion of a TGF-beta receptor.

8. The method of claim 1, wherein the inhibitor of TGF-beta activity comprises a bi-specific antibody that (i) (a) binds TGF-beta or (b) binds a TGF-beta receptor, and (ii) also binds a human transferrin receptor.

9. The method of claim 1, wherein the inhibitor of TGF-beta activity comprises (i) a monoclonal anti-TGF-beta antibody conjugated to a lipoprotein receptor related protein receptor (LRP-1) binding-peptide of 8-40 amino acids, or (ii) a monoclonal anti-TGF-beta receptor antibody conjugated to a lipoprotein receptor related protein receptor (LRP-1) binding-peptide of 8-40 amino acids.

10. The method of claim 1, wherein the inhibitor of TGF-beta activity is a synthetic small organic compound.

11. The method of claim 1, wherein the TGF-beta activity being inhibited is TGF-beta1 activity, TGF-beta2 activity or TGF-beta3 activity.

12. The method of claim 1, wherein the administration of the amount of an inhibitor of TGF-beta activity does not significantly decrease systemic circulation TGF-beta levels in the subject.

13. The method of claim 1, wherein the subject is clinically obese.

14. The method of claim 1, wherein the subject's age is 40 years or older.

15-17. (canceled)

18. A method of reducing glucose intolerance in a subject comprising administering to the subject an amount of an inhibitor of TGF-beta activity, in a manner effective to enter the central nervous system (CNS) of a subject, effective to reduce glucose intolerance in a subject.

19. (canceled)
